(19) United States
(12) Patent Application Publication
(10) Pub. No.: US 2002/0028986 A1
(43) Pub. Date: Mar. 7, 2002

(54) Light source for use with scopes

(76) Inventor: Robert Lee Thompson, Rogers, AR (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/945,987

(22) Filed: Sep. 4, 2001

Related U.S. Application Data
(60) Provisional application No. 60/230,658, filed on Sep. 7, 2000.

(51) Int. Cl. A61B 1/06

(52) U.S. Cl. 600/178

(57) ABSTRACT

A light source for use with a scope is provided. The scope includes a body having a proximal end and a distal end, a light input port disposed on the body, a light output port disposed on the body and disposed distally of the light input port and a light transmission channel extending between the light input port and the light output port. The light source includes a housing including a light output port and a light source disposed within the housing. The housing is adapted to mount to the body of the scope to provide light to the scope.

### CROSS-REFERENCE TO RELATED APPLICATIONS

This application claims the benefit of U.S. Provisional Patent Application No. 60/230,658, filed Sep. 7, 2000.

### BACKGROUND OF THE INVENTION

1. Field of the Invention

The present invention relates to light sources and, more particularly, to light sources for use with scopes.

2. Related Art

Scopes are known for use in peering into cavities that are not conveniently accessible. For example, endoscopes are known for visualizing body parts within a human body cavity and within cavities of other animals, and bore scopes are known for visualizing components within cavities of machinery, such as aircraft engines. Whether for use in medical or non-medical fields, such scopes typically are either rigid, flexible or otherwise positionable.

An illustrative scope 10 is shown in FIG. 1. Scope 10 includes an elongated body 12 having a probe 14 on a distal end for insertion or probing into a cavity 15 and an eyepiece 16 at the proximal end. Typically, the scope 10 can be used with a camera system (not shown) that attaches, through a coupler (not shown), to the eyepiece 16 and enables images to be captured. The body 12 may also include a light post 18, in optical communication with a light transmission channel 19 extending to the distal end 14, that is adapted to couple to a light source 20. The scope typically also includes a fiber optic cable (not shown) that is disposed internally within the light transmission channel 19 and relays light from the light post 18 to the probe 14 to illuminate the cavity 15.

The light source 20 typically is an expensive, heavy (e.g., 5 pounds or more), bulky and cumbersome apparatus that must be plugged into a power outlet. The light source 20 has a relatively large light box 22 including, for example, a light bulb (not shown). Examples of typical light bulbs include halogen or xenon light bulbs that generate significant heat. A fiber optic cable 24 is attached at one end to the light box 22 and at another end to the light post 18 of the scope 10 via a coupler 26. The fiber optic cable 24 transmits light from the light box 22 to the scope 10.

### SUMMARY OF THE INVENTION

In one embodiment, an apparatus includes a scope and a light source assembly. The scope includes a body having a proximal end and a distal end, a light input port disposed on the body, a light output port disposed on the body and disposed distally of the light input port and a light transmission channel extending between the light input port and the light output port. The light source assembly includes a housing having a light output port. The housing is mounted to the body with the light output port of the housing in optical communication with the light input port on the body. At least one light source is disposed within the housing.

In another embodiment, a light source assembly for use with a scope is provided. The scope includes a body having a proximal end and a distal end, a light input port disposed on the body, a light output port disposed on the body and disposed distally of the light input port and a light transmission channel extending between the light input port and the light output port. The light source assembly includes a housing having an interface to mount the housing to the body of the scope. The housing further includes a light output port that is adapted to mate with the light input port on the body when the interface engages with the body. The light source assembly also includes at least one light source disposed within the housing.

In another embodiment, a coupler for coupling a light source assembly to a scope is provided. The scope includes a body having a proximal end and a distal end, a light input port disposed on the body, a light output port disposed on the body and disposed distally of the light input port, and a light transmission channel extending between the light input port and the light output port. The light source assembly includes a housing having a light output port and at least one light source disposed within the housing. The coupler includes a coupler body, first and second interfaces formed on the coupler body, and a light transmission channel in optical communication with the first and second interfaces. The first interface is adapted to be mounted to the light source assembly and aligned with the light output port on the light source assembly. The second interface is adapted to be mounted to the scope and aligned with the light input port on the scope. Light transmission from the light source is transmitted to the scope through the interface.

In yet another embodiment, a camera system for use with a light source assembly and a scope is provided. The scope includes a body having a proximal end and a distal end, a light input port disposed on the body, a light output port disposed on the body and disposed distally of the light input port and a light transmission channel extending between the light input port and the light output port. The light source assembly includes a housing including a light output port and at least one light source disposed within the housing. The camera system includes a camera and a camera controller, coupled to the camera, to control the camera. The camera controller includes an interface adapted to further couple to the light source assembly. The camera controller further is adapted to control the light source assembly.

In another embodiment, a camera coupler for use with the scope is provided. The scope includes a body having a proximal end and a distal end, a light input port disposed on the body, a light output port disposed on the body and disposed distally of the light input port, a light transmission channel extending between the light input port and the light output port, and a view port. The camera coupler includes a coupler body adapted to couple the camera to the view port and a light source assembly connected to the coupler body and adapted to couple with the light input of the scope.

In still another embodiment, a method for providing light to a scope is provided. The method includes the act of providing a scope having a body, the body having a proximal end and a distal end, a light input port disposed on the body, a light output port disposed on the body and disposed distally of the light input port and a light transmission channel extending between the light input port and the light output port. The method also includes the acts of providing a light source assembly having a housing, including a light output port, and a light source disposed within the housing, and mating the light source assembly to the scope with the light output port of the light source assembly aligning with the light input port of the scope.

Various embodiments of the present invention provide certain advantages and overcome certain drawbacks of prior light sources. Embodiments of the invention may not share the same advantages, and those that do may not share them under all circumstances. This being said, the present invention provides numerous advantages including increased portability and ease of use of the light source.

Further features and advantages of the present invention, as well as the structure of various embodiments, are described in detail below with reference to the accompanying drawings.

### BRIEF DESCRIPTION OF THE DRAWINGS

Various embodiments of the invention will now be described, by way of example, with reference to the accompanying drawings, in which:

FIG. 1 is a perspective view of a prior art scope and light source system;

FIG. 2 is a perspective view of a scope with a light source assembly according to one embodiment of the invention;

FIG. 3 is a perspective view of a scope with light source assembly according to another embodiment of the invention;

FIG. 4 is a perspective view of a scope with light source assembly according to yet another embodiment of the invention;

FIG. 5 is a partially cut away perspective view of a light source assembly according to a further embodiment of the invention;

FIG. 6 is a perspective view of a camera assembly and light source assembly for use with a scope according to another embodiment of the invention;

FIG. 7 is a partially cut away perspective view of a coupler and imaging unit according to another embodiment of the invention;

FIG. 8 is a partially cut away perspective view of the coupler and the imaging unit shown in FIG. 7;

FIGS. 9a and 9b are partially cut away perspective views of an illustrative focusing mechanism employed in the system of FIGS. 7-8;

FIG. 10 is a partially cut away perspective view of an alternative embodiment of the invention directed to an imaging system including an adapter that adapts a standard camera head to be mated with the coupler shown in FIGS. 7-8;

FIG. 11 is a partially cut away perspective view of the adapter shown in FIG. 10; and

FIG. 12 is a perspective view of a camera assembly and light source assembly according to another embodiment of the invention.

### DETAILED DESCRIPTION

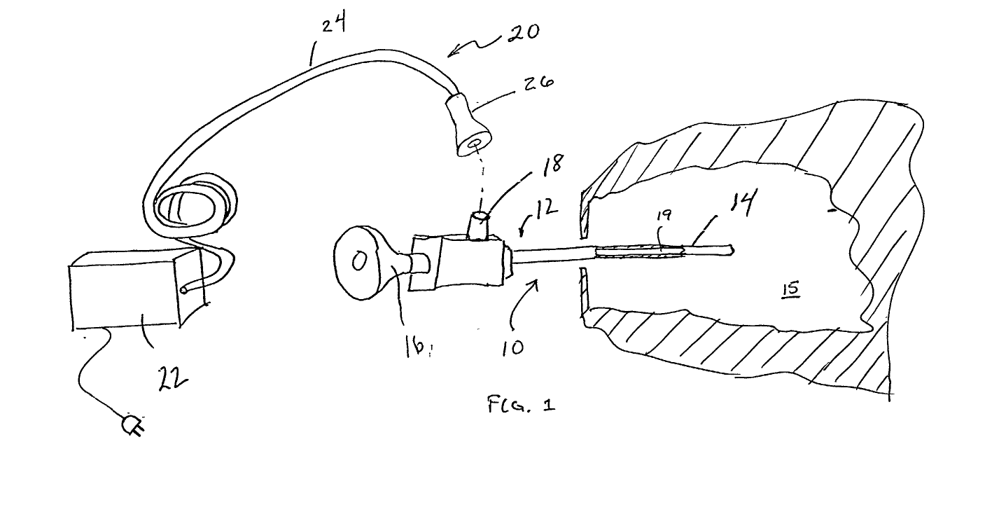

The Applicant has found certain disadvantages with prior art light sources attached to scopes. For example, the typically long fiber optic cable 24 shown in FIG. 1 impedes the maneuverability of the scope 10 and mobility of the entire system, as the fiber optic cable may become entangled with itself or other objects as the scope is moved into position. Further, the relatively heavy and large light box is undesirable because of the need to carry it along and move it into various locations, resulting in a system that is not easily portable. In addition, power to the light box is provided through a conventional power cord that plugs into an electrical outlet, further resulting in a system that is not easily portable. This is especially inconvenient when used in conjunction with scopes for use in non-medical fields where the system may need to be carried around or be used in locations remote from a power outlet.

One embodiment of the invention is directed to a light source for use with a scope. The light source may be mounted to the scope resulting in increased maneuverability of the scope and mobility of the entire system. The light source may be relatively light weight, enabling it to be easily carried along and moved into various locations, resulting in a system that is easily portable. In addition, the light source may include a portable power source, thereby rendering the light source cordless, further resulting in a system that is easily portable. This may be especially convenient when used in conjunction with scopes for use in non-medical fields where the system may need to be carried around or be used in locations remote from a power outlet. It is to appreciated, however, that the light source of the present invention may be used with scopes for medical use as well as with scopes for non-medical uses.

The light source assembly may be useful with scopes used to visualize any number of objects such as engines and related components, including aircraft engines, ship engines, motor vehicle engines and turbine engines; structural components of vehicles, such as airframes, hulls, chassis and automobile frames and other such components; and facilities, such as manufacturing plants, nuclear power plants, and buildings and structures. Other applications for the scope include, but are not limited to, cargo inspecting by customs agents; searching by law enforcement officials and military personnel; and imaging and inspecting for the space industry, the entertainment sports and recreation industry, and the medical field. Other applications will be readily apparent to those of skill.

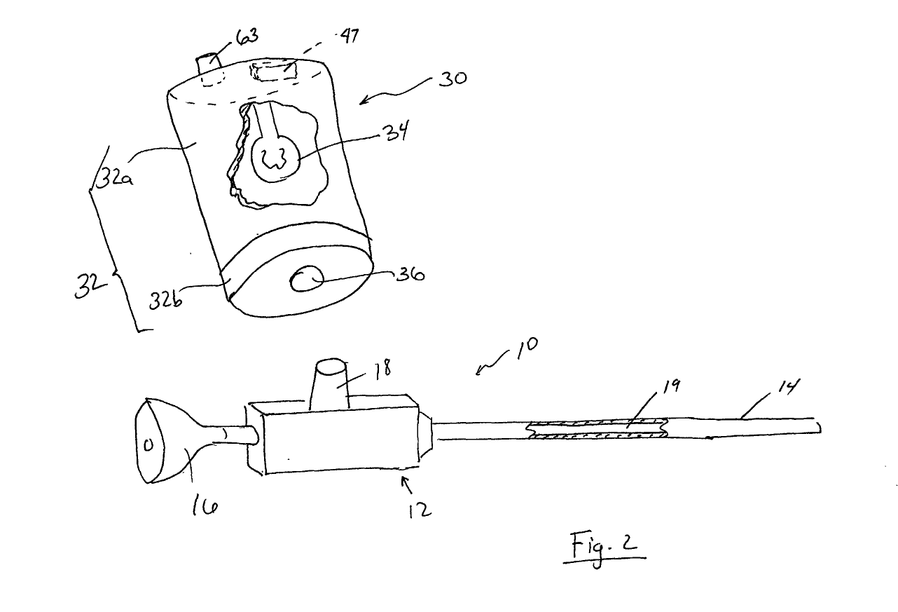

In one embodiment, a light source is directly coupled to the scope rather than through a fiber optic cable. This can be done in numerous ways. For example, in one embodiment shown in FIG. 2, a light source assembly 30 includes a housing 32 that has a suitable light source 34 disposed therein. As used herein, light source means a device that actually generates light rather than convey light generated by another device (e.g., the way a fiber optic cable conveys light). The housing 32 is configured to mate, via an aperture 36, with a light post 18 of a conventional scope 10. In this manner, the light source assembly is a stand-alone unit. The light source assembly may be removably attached directly to any suitable scope, as depicted in FIG. 2, using any suitable technique, such as screwing, snapping or merely resting on the light post, as the present invention is not limited to any particular attachment techniques. Once the light source assembly is coupled to the light post, light may be transmitted to the light transmission channel 19 of the scope 10. As discussed above, the light transmission channel may include a fiber optic cable or other such medium to transmit light through the scope. However the present invention is not limited in this respect. As such, the scope 10 may employ any suitable technique, such as fluids, lenses, prisms, mirrors, a hollow tube or other such channel, etc. to transmit light through the scope.

The light source assembly 30 may be adapted to transmit light in a manner that reduces or eliminates light from otherwise escaping the housing. For example, the housing may be formed with an opaque material. Alternatively, the housing 32, or the light source 34, may include a shroud (not shown) that allows light to be transmitted in one or more desired directions.

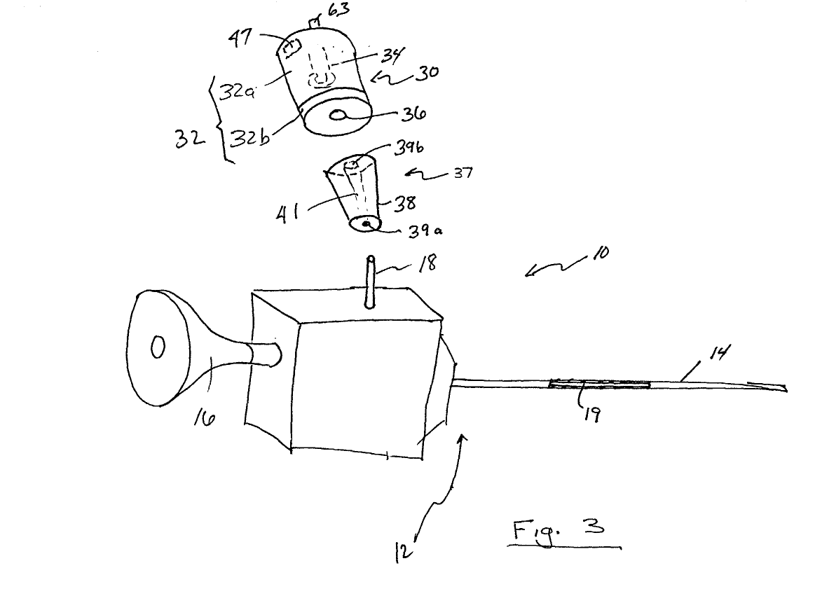

In one embodiment, the light source assembly 30 may be removably attached to the scope through an interface or coupler 37, as shown in FIG. 3. The coupler may be used to so that the light source assembly 30 may be attached to scopes having light posts of varying sizes or configurations. For example, as shown in FIG. 3, the coupler 37 includes a body 38 having a first receptacle or interface 39a on one end of the body that can mate with the particular light post 18 employed on the scope 10. In the example shown in FIG. 3, the light post 18 is smaller than the aperture 36. Another end of the coupler 37 includes a second receptacle or interface 39b that can mate with the particular aperture 36 employed on the light source assembly 30. The coupler may be attached to the light post and aperture using any suitable technique, such as screwing, snapping or merely resting thereon. A light transmission channel 41 that communicates with the receptacles 39a and 39b may be used to transmit light through the coupler 37 so that light may be received by the light post 18 from the light source assembly 30. The light transmission channel 41 can be implanted in any of a number of ways, such as with the use of fluids, lenses, prisms, mirrors, a hollow tube or other such channel to transmit light through the coupler.

It should be appreciated that although the coupler 37 is shown with one receptacle being smaller than the other, the invention is not limited in this respect. Rather, the coupler 37 may include any suitably sized or shaped receptacles, such that one receptacle can mate with the light source assembly 30 and the other can mate with the light post. Also, although the coupler 37 is shown as having female receptacles, the present invention is not limited in this respect as other styles of interfaces may be employed.

In one embodiment, a plurality of couplers 37 may be provided, each configured to mate with a certain style scope 10. In this manner a user may have flexibility in selecting a particular scope without being hindered by the configuration of the light source assembly or of the scope. Also, when a coupler 37 is employed, the light source assembly 30 need not have an aperture 36 to mate with the light post 18. Rather, the coupler 37 may be mated with the light source assembly in any suitable manner.

As described above with respect to the light source assembly, the coupler 37 may also cause light to be transmitted directly to the light post without otherwise allowing light to escape from the coupler. This may be accomplished in any suitable way including, for example, forming at least portions of the coupler with an opaque material or placing a shroud around at least a suitable portion of the coupler.

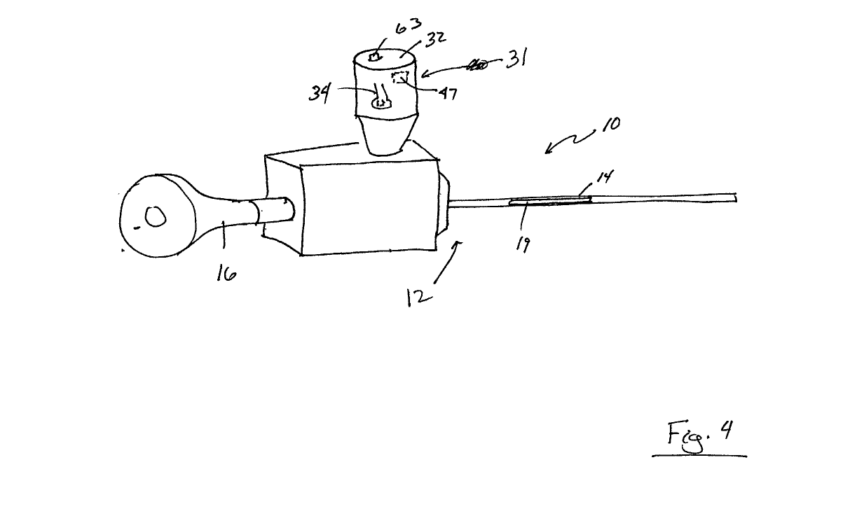

It should be appreciated that the present invention is not limited to coupling the light source assembly directly or indirectly to the scope. For example, a light source assembly 31 may be integrally formed with the scope such that the scope and light source are supplied as a single unit, as shown in FIG. 4. In addition, the light source may be a separate unit or it may be integrally formed with any other component of the system, such as the camera or the coupler that couples the camera to the scope, as will be explained below.

The light source 34 within the light source assembly may be any suitable light-emitting device as aspects of the present invention are not limited in this manner. In one embodiment, the light source is one or more (e.g., seven) light-emitting diodes (LEDs). LEDs may be advantageous due to their low cost, low power consumption and low heat generation. Alternatively, halogen, xenon, incandescent or other light sources may be used.

The housing 32 of the light source assembly may provide access to the light source so that it can be replaced should it burn out. In one embodiment, the housing 32 comprises a main body portion 32a (see FIGS. 2-3) and a removable cap 32b. The cap 32b may be threaded onto the body portion 32a, may be snapped onto the body portion 32a, or may employ other suitable techniques for removably attaching the lid 32b to the body 32a, as the present invention is not limited in this respect. Examples of other such techniques include employing hinges, latches, hook and loop fasteners, etc. Further, other suitable techniques for providing access to the light source may be employed, such as a door formed in the side of the housing 32, or a removable plug containing the light source formed, e.g., at the rear of the housing.

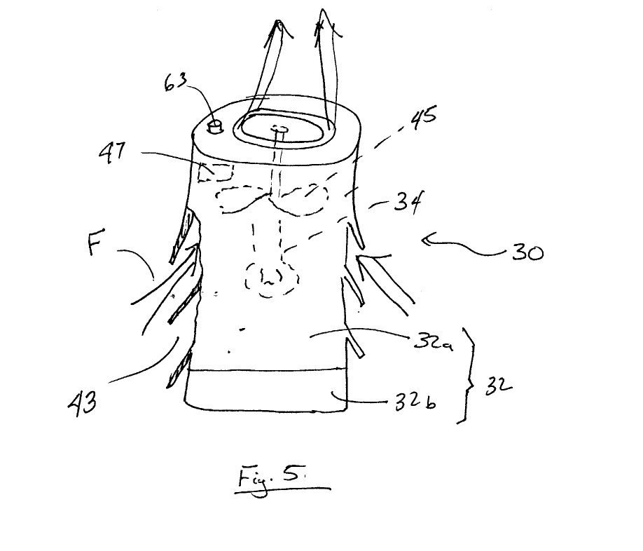

When using a light source that generates significant heat, it may be desirable to adequately vent the housing so that the light source assembly does not overheat, as it may be disposed adjacent other system components. This may be accomplished in any suitable manner, including, but not limited to convection or radiant cooling. In one embodiment, vents 43 are formed in the housing, as shown in FIG. 5. The vents allow air flow F through the housing 32 to cool the light source assembly. In addition, or in the alternative, a fan 45 may be used to actively cool the light source assembly. The fan may be disposed within the housing, as shown, or may be placed outside the housing.

Power to the light source may be provided through conventional power cords, as some aspects of the invention are not limited in this respect. However, in one embodiment, power is provided via a portable power source (e.g., a battery 47 in FIG. 4) to create a cordless, portable system. If the light source is powered by a battery or other portable power source, the power source may be disposed within the housing 32, as shown, contained within a power pack that can be attached directly to the housing 32 or otherwise through an indirect connection, or disposed elsewhere in the system and coupled through a power cord, as will be described below, still resulting in a cordless system. In this respect, reference to a cordless system means a power source, such that plugging into a power outlet is not necessary, and does not preclude the use of cords or connectors between the power source and the light source assembly or the use of an on-board power source.

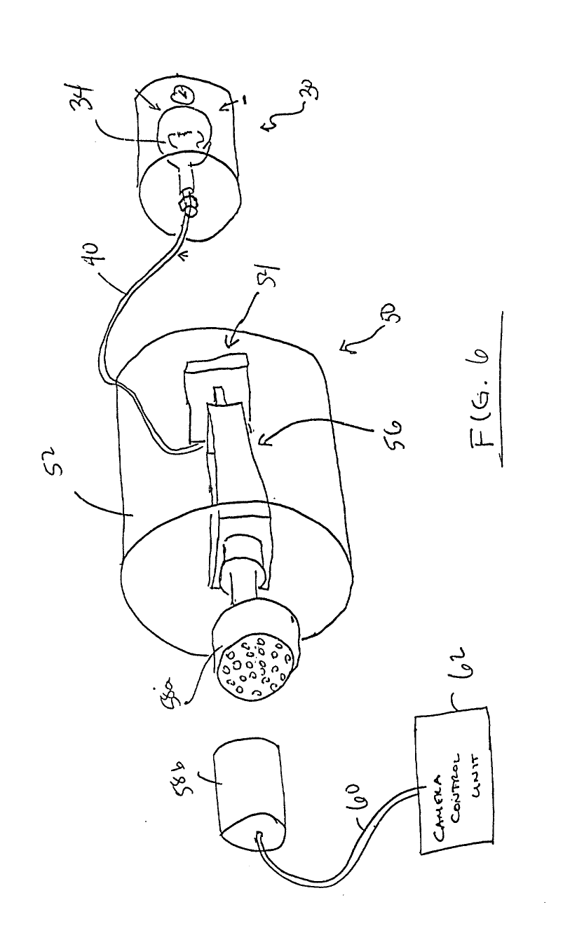

The light source assembly 30 may communicate with a camera assembly 50, also referred to as an imaging unit, through a cord 40, as shown in FIG. 6. In this embodiment, the camera assembly 50 is configured to mount to the scope 10 with or without a suitable coupling device, as will be explained below. The scope may include or otherwise attach to a C, S, D, or V mount, or any other suitable mount, as the present invention is not limited in this respect. Power may be supplied by the camera assembly to the light source assembly 30. With a camera assembly that is portable and cordless, the entire system is portable and cordless, despite a cord between the camera assembly and the light source assembly.

The present invention is not limited to the details of the camera assembly 50 and can be used with any camera, or with no camera. A typical camera assembly 50 includes a housing 52 having a charge couple device 54 (CCD) disposed therein for converting images to digital signals. The CCD communicates with suitable electronics 56, which, in turn, relay the signals through connector portions 58a and 58b and cord 60 to a camera control unit 62. The camera may compensate for the intensity of light supplied by the light source with the use of an electronic iris, as in conventional cameras for use with scopes. The camera control unit 62 may be used to control the camera in any suitable manner. The camera control unit may also be used to display, transmit and/or store data and/or images to one or more local or remote locations, as desired, for subsequent viewing and/or storage. The camera control unit 62, which itself may be powered by one or more portable power sources (e.g., batteries), may also supply power to both the camera assembly 50 and, via cord 40, the light source assembly 30. The camera (e.g., the camera control unit 62) may control one or more operating characteristics of the light source (e.g., on/off, intensity). Alternatively, switching of the light source on or off or controlling the intensity may be accomplished with one or more suitable switches 63 (FIGS. 2-5) mounted on the housing 32 or elsewhere, as the present invention is not limited in this respect. For embodiments that are not portable, power may be supplied through conventional power cords, either directly to the light source assembly 30 or via the camera.

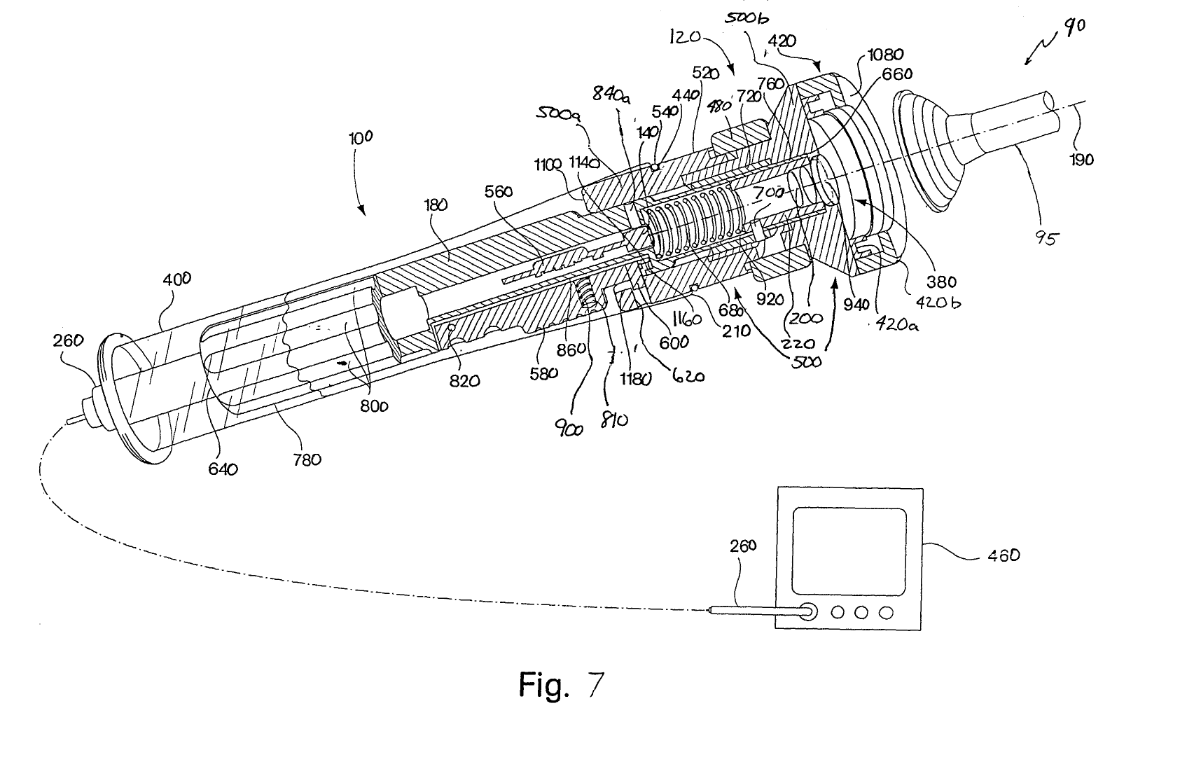

One example of an imaging system, including a camera assembly and scope, with which the light source assemblies of the present invention can be used will now be described with reference to FIGS. 7-11. However, it is to be appreciated that the light source assemblies of the present invention are not limited to use with this or any other particular camera assembly and/or scope.

FIG. 7 is a partially cut away perspective view of an example of an imaging system that may be used with the light source assemblies of the invention. As shown, the imaging system includes four primary components, i.e., a scope 90, such as an endoscope, an imaging unit or camera assembly 100, a coupler 120, which couples the scope 90 to the imaging unit 100, and a condom-like drape 400, which prevents the imaging unit 100 from contaminating a sterile operating field should the system be used in a medical application. The imaging system can be employed with any type of image-producing scope, and is not limited to use with any particular type of scope.

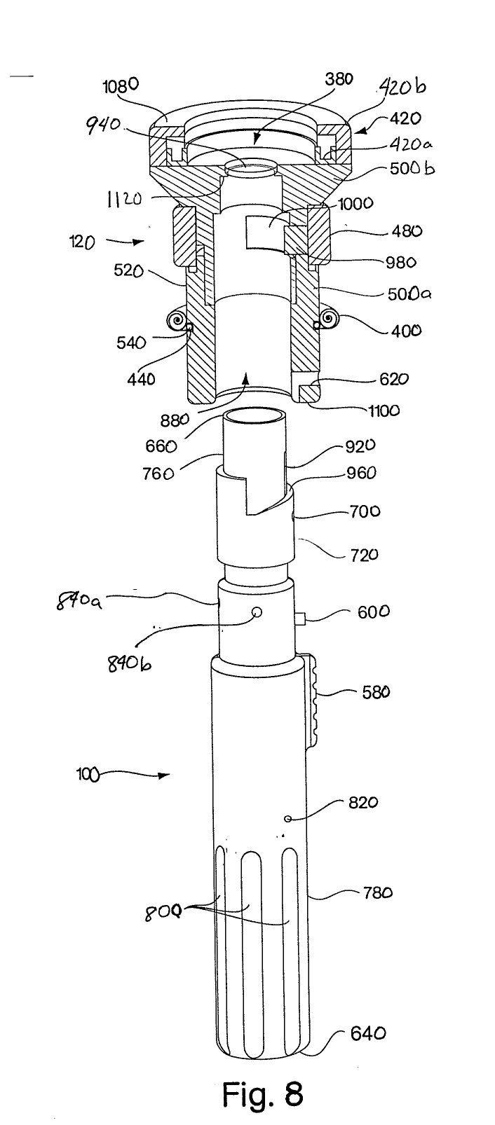

As discussed in more detail below, in the exemplary imaging system shown in FIGS. 7-8, the condom-like drape 400 does not intercept the optical viewing axis of the system. In addition, the condom-like drape 400 does not cover a focusing mechanism 480 of the imaging system, making it easier to focus the system and lessening the likelihood that the drape 400 will be damaged due to manipulation of the focusing mechanism.

The lens for focusing the image from the endoscope to the imaging unit may be provided in the imaging unit 100, rather than in the coupler 120. This is particularly advantageous because, as discussed in more detail below, in the exemplary embodiment shown, a portion of the coupler 120 is not separated from the scope 90 by the condom-like drape 400, and therefore, is sterile in use. By removing the refractive lens 200 from the coupler 120, the coupler 120 can be made significantly less expensively, thereby enabling the coupler 120 to be provided as a disposable part that need not be sterilized between uses. This is advantageous because the sterilization of the devices can be inconvenient and time consuming.

The imaging unit 100 includes an image sensor 140 that senses an image along an imaging axis (not shown). When the imaging system is used, the coupler 120 is coupled between the eyepiece 95 of the scope 90 and a distal end 660 of the imaging unit 100 such that the lens 200 is disposed between the image sensor 140 and the eyepiece 95 to focus an image produced by the scope 90 onto the image sensor 140. The refractive lens 200 may be provided in the imaging unit 100, rather than in the coupler 120. The coupler can be therefore made significantly less expensively, thereby enabling the coupler to be provided as a disposable part that need not be sterilized between uses.

The image sensor 140 may, for example, include a charge-coupled device (CCD) as discussed above with reference to FIG. 6, or a metal-oxide semiconductor (MOS) sensor. It should be appreciated, however, that the present invention is not limited in this respect, and can be employed with any type of image sensor 140. The image generated by the image sensor 140 can be conveyed to a monitor 460 in any of numerous ways, and the present invention is not limited to any particular implementation. For example, the image sensor 140 may be coupled to circuitry 560 which can assist in converting an image sensed by the image sensor 140 into an electrical signal. This electrical signal then may be transmitted (e.g., via cable 260) to the monitor 460 or elsewhere for display to a user or may be otherwise processed and/or recorded on a suitable medium. Alternatively, the image sensor 140 may comprise a bundle of fiber optic cables which optically transmit an image from the lens 200 to a viewing device for display to a user. Thus, the image sensor 140 need not necessarily convert the image from scope 90 into an electrical signal.

The imaging unit 100 is releasably mated with the coupler 120. This mating may be accomplished using any of a number of techniques. FIGS. 7 and 8 illustrate one technique that may be used to mate these two components. In the particular implementation shown, to mate imaging unit 100 with coupler 120, a distal end 660 of the imaging unit 100 is inserted into an opening 880 at a proximal end 1100 of the coupler 120. As shown, the imaging unit 100 includes a button 580 which is pivotally connected, via a pin 820, to a body portion 180 of the imaging unit 100. The imaging unit 100 has a cavity 810 formed underneath the button 580 and a spring 900, disposed in the cavity 810. Spring 900 biases the button 580 (in a clockwise direction in FIG. 7) about pin 820 so that locking member 600 is biased away from a surface 860 of body portion 180. When a user pushes button 580 toward surface 860, however, spring 900 is compressed so that button 580 moves in a counterclockwise direction in FIG. 7 about pin 820 and locking member 600 moves toward surface 860. Thus, when the button 580 is depressed and the distal end 660 of the imaging unit is inserted into the opening 880 in the coupler 120, the locking member 600 moves toward surface 860 so that it can slide over edge 1180 of the coupler 120. When the button 580 is released, the locking member 600 is biased (by spring 900) away from surface 860 and into a notch 620 in the coupler 120, and a shoulder 1160 of imaging unit 100 contacts a shoulder 1140 of the coupler 120, thereby interlocking the imaging unit 100 and the coupler 120. An indication that the distal end 660 of the imaging unit 100 is fully inserted into the opening 880 is provided by the distal end 660 contacting a shoulder 1120 of coupler 120. The imaging unit 100 and coupler 120 can be separated by pushing button 580, which moves the locking member 600 out of the notch 620, and pulling the imaging unit 100 away from the coupler 120. As mentioned above, FIGS. 7 and 8 illustrate only one example of the many ways that the imaging unit 100 and coupler 120 may be mated together.

As shown in FIGS. 7 and 8, the imaging unit 100 also includes a handle 780 proximal to the body portion 180. The handle 780 may include grooves 800 to make it easier for a user to grip the imaging unit 100 though the drape 400 that can be extended over the imaging unit 100 in a manner described below.

The image sensor 140 and circuitry 560 may be mounted in the body portion 180 of the imaging unit 100 in any of a number of ways. For example, the image sensor 140 may be mounted via pins or screws 840a and 840b, and circuitry 560 may be mounted on a circuit board supported within body portion 180. One or more wires (not shown) may be used to interconnect the circuitry 560 with the cable 260.

It may be useful to enable the focal length between the image sensor 140 and the lens 200 of imaging unit 100 to be adjusted. In the system shown in FIGS. 7-8, this is accomplished via a mechanism that is not covered by the condom-like drape 400, thereby making it easier to focus the system and lessening the likelihood that the drape 400 will be damaged due to manipulation of the focusing mechanism. It should be appreciated, however, that the focal length adjustment can be accomplished in any number of ways.

One example of a technique that is useful to perform the focal length adjustment is illustrated in FIGS. 7-9. In the embodiment shown, the refractive lens 200 is disposed in the imaging unit 100, rather than in the coupler 120. Thus, the focusing mechanism includes elements disposed in the imaging unit 100, as well as in the coupler 120. As mentioned above, placement of the lens 200 within the imaging unit 100, rather than in the coupler 120, provides at least one significant advantage. That is, the cost of the coupler 120 may be reduced significantly below the cost of coupling devices that include lenses, thereby making it commercially practicable to use a new, sterile coupler each time the imaging system is used, rather than repeatedly sterilizing and reusing the same coupling device.

The distal end 660 of the imaging unit 100 includes a primary cylinder 760, in which a spring 680 and a cylindrical lens holder 220 are disposed. Lens holder 220 supports the lens 200 in front of an imaging axis of image sensor 140. Lens holder 220 (and lens 200) can be moved within primary cylinder 760 either toward or away from distal end 660 of the imaging unit 100 so as to adjust the focal length between the image sensor 140 and the lens 200. Spring 680 biases lens holder 220 toward distal end 660. The position of lens holder 220 within primary cylinder 760 can be adjusted, however, through manipulation of a focusing mechanism on the coupler 120 as discussed below.

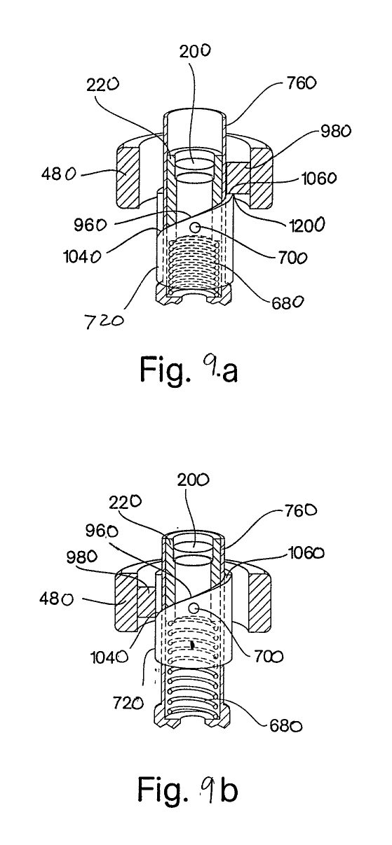

The imaging unit 100 further includes an outer cylinder 720, including a spirally ramped upper edge 960, which surrounds the primary cylinder 760. Outer cylinder 720 is movable with respect to primary cylinder 760 either toward or away from the distal end 660 of imaging unit 100. Outer cylinder 720 is connected to the lens holder 220 via a pin 700. Pin 700 extends through a slot 920 which extends a short distance along a length of the primary cylinder 760. Thus, lens holder 220, outer cylinder 720 and pin 700 move as a single unit, with respect to primary cylinder 760, either toward or away from the distal end 660 of imaging unit 100. The manner in which this unit interacts with the focusing mechanism disposed on coupler 120 is described below in connection with FIGS. 9a-9b.

FIGS. 7 and 8 show an exemplary implementation of the coupler 120. The coupler 120 can be constructed in any of a number of ways to achieve the desired goal of enabling the imaging unit 100 to be coupled to the scope 90. In the implementation shown, the coupler 120 includes a main body 500 (including a proximal portion 500a and a distal portion 500b), a focusing ring 480, a light-penetrable window 940, a scope mounting portion 420 (including inner ring 420a and outer ring 420b) and the condom-like drape 400. The components constituting the main body 500, focusing ring 480 and scope-mounting portion 420 may be made of any suitable material and may be affixed together in any suitable manner. For example, they may be plastic molded components affixed together using an epoxy-based adhesive. When the coupler 120 is a disposable device, the coupler 120 is preferably formed from inexpensive components.

The main body 500 may be formed by inserting the distal portion 500b within the focusing ring 480, and then affixing together the proximal and distal portions 500a and 500b. Scope mounting portion 420 may be affixed to distal portion 500b. Main body 500 has an outer surface 520 between a distal end 1080 and a proximal end 1100 of the coupler 120. A channel 440 extends about a perimeter of the outer surface 520 between the focusing ring 480 and the proximal end 1100.

When the coupler 120 is used in a medical application, it is generally important that the environment to which the patient is exposed remains sterile. It is also desirable, however, to not have to sterilize the imaging unit 100, thereby saving the time and expense of sterilization, and avoiding restrictions on the manner in which the imaging unit be formed, since it need not be sterilizable. Therefore, a sterile barrier may be established between the sterile operating environment including the scope 90, and a non-sterile environment including the imaging unit 100. In the system shown in FIGS. 7-8, such a sterile barrier is established by coupling the distal end 660 of the imaging unit 100 to the coupler 120, and providing a hermetic seal between the components of the coupler 120 that separate the sterile and non-sterile environments. A light-penetrable window 940 is hermetically sealed between the distal end 1080 and the proximal end 1100 of the coupler 120 to establish a sterile barrier therebetween. Window 940 may be made of glass, plastic, or any other suitable material through which light can pass from the scope 90 to the image sensor 140 (via lens 200) to generate a suitable image.

As mentioned above, the coupler 120 also includes the condom-like drape 400. The condom-like drape 400 may be made of any material that is suitable for creating a sterile barrier between a sterile environment and a non-sterile environment. For example, the condom-like drape may be made of a non-porous latex or plastic material. When the imaging unit 100 is mated with the coupler 120, the drape 400 may be extended to cover some or all of imaging unit 100 and cable 260 (FIG. 2). The condom-like drape 400 may be hermetically sealed to the outer surface 520 of coupler 120. It should be appreciated that in the implementation shown in the figures, when each of the components of the coupler 120 is sterile, the hermetic seals between the main body portion 500 and the window 940 and drape 400 establish a sterile barrier between the scope 90 and the imaging unit 100, with the main body portion 500 of the coupler 120 itself forming a part of this sterile barrier. As compared to other systems, in which a sterile barrier is formed only with a drape and a window portion thereof and in which a coupling device is located entirely on the non-sterile side of this barrier, the system shown in FIGS. 7 and 8 is superior because scope 90 can mate directly with body portion 500 rather than requiring the drape to be interposed between the coupling device and the endoscope.

In the system shown in the figures, the condom-like drape 400 does not intercept the optical viewing axis 190 of the imaging system. As mentioned above, this is advantageous in that the drape 400 need not be provided with a window that must be aligned with the optical viewing axis 190, and the drape 400 does not interfere with the quality of the image presented on the monitor 460. It should be appreciated that the function performed by the condom-like drape 400 can be achieved in any of numerous ways. For example, a protective drape can be provided that is more rigid than the condom-like drape 400 depicted in the drawings.

In the system shown in the drawings, the condom-like drape 400 is substantially tubular in form and is open on its distal and proximal ends. The distal end 210 of the condom-like drape 400 is attached to the outer surface 520 (within channel 440) of the coupler 120. As discussed above, this attachment can be accomplished using a hermetic seal (e.g., via an O-ring 540) to maintain the separation between the sterile and non-sterile environments. The condom-like drape 400 can be provided in a rolled-up form attached to the coupler 120. After the coupler 120 is mated with to the imaging unit 100 as described above, the condom-like drape 400 can be unrolled to cover the non-sterile imaging unit 100. By encompassing the outer surface 520 of coupler 120 with the opening at the distal end 210 of the drape 400, the drape 400 can be used in conjunction with coupler 120 without requiring the user to align the drape 400, or a window portion thereof, between the eyepiece 95 of the scope 90 and the coupler 120, and without having the drape 400 intercept the optical viewing axis 190 of the imaging system.

FIGS. 7 and 8 illustrate one example of a technique that may be used to mate the scope 90 with the coupler 120. It should be appreciated that numerous other suitable mating techniques can be employed. In the system shown in FIGS. 7 and 8, the scope 90 is mated with the coupler 120 by inserting the eyepiece 95 into an opening 380 at the distal end 1080 of the coupler 120. Opening 380 may be formed by the inner and outer rings 420a-420b of the scope mounting portion 420. The inner and outer rings 420a-420b form equal diameter openings, and inner ring 420a is movable with respect to outer ring 420b. A spring biases the inner ring 420a so that its center is forced to be offset from the center of the outer ring 420b unless a user activates a lever (not shown) to cause the centers of the two rings to align with one another.

To mate the scope 90 with the coupler 120, the user activates the lever so that the centers of the rings 420a-420b align with one another and inserts the eyepiece 95 through both rings. The user then can release the lever so that the spring (not shown) causes the center of ring 420a to become offset from the center of ring 420b. Because the diameter of the eyepiece 95 is only slightly smaller than the diameter of each of rings 420a and 420b, when the centers of the rings are offset from one another, the eyepiece 95 will be locked within the scope mounting portion 420 of the coupler 120. The eyepiece 95 may be separated from the scope mounting portion 420 by pressing the lever to realign the centers of rings 420a and 420b and pulling the scope 90 away from the coupler 120.

In the system of FIG. 7, the coupler 120 is shown as being mated directly with the eyepiece 95 of the scope 90. However, it should be appreciated that the scope 90 (or other image-producing scope) may alternatively be mated indirectly with the coupler 120. For example, the scope 90 may be mated with the coupler 120 via one or more additional coupling devices.

As discussed above, using the system of FIGS. 7-9, the user can directly manipulate a focusing mechanism without having to do so through a portion of a protective drape such as condom-like drape 400. Any focusing mechanism can be employed that serves to adjust the focal length between the lens 200 and image sensor 140 in the imaging unit 100. In the exemplary system shown in FIGS. 7-9, a focusing ring 480 is provided on the coupler 120 to perform this focal length adjustment. The focusing ring 480 is disposed distally of the distal end 210 of the condom-like drape 400, so that after the drape 400 is extended to cover some or all of the imaging unit 100 and cable 260 (FIG. 7), the focusing ring 480 is not covered by the drape 400 and may be manipulated by a user to adjust the focal length between the lens 200 and the image sensor 140 without also having to manipulate the drape 400. Hence, this feature makes focusing ring 480 relatively easy for the user to manipulate to achieve sharp focusing, and reduces the risk of damage to drape 400.

An illustrative example of a linkage assembly for mechanically coupling the focusing ring 480 on the coupler 120 to the imaging unit 100 to adjust the focal length between the lens 200 and image sensor 140 is shown in FIGS. 8, 9a and 9b. It should be appreciated that numerous other implementations are possible. In the system shown, the distal portion 500b of the main body portion 500 of coupler 120 has an annular groove 1000. Annular groove 1000 may be covered by the focusing ring 480, so that it is not visible from the outside of coupler 120. A finger 980 extends inwardly from the focusing ring 480 through the annular groove 1000, so that when the focusing ring 480 is rotated about the main body portion 500, finger 980 slides within the annular groove 1000.

As shown in FIGS. 9a and 9b, when the imaging unit 100 is mated with the coupler 120, a lower surface 1200 of finger 980 contacts a portion of a spiraling ramp surface 960 on the outer cylinder 720. As mentioned above, pin 700 may be connected between the outer cylinder 720 and the cylindrical lens holder 220 through the slot 920, which extends along the length of the primary cylinder 760, so that the outer cylinder 720 and lens holder 220 do not rotate with respect to the primary cylinder 760. The focusing ring 480, however, can rotate freely about the primary cylinder 760, limited only by the movement of the finger 980 within the annular groove 1000.

As the focusing ring 480 rotates with respect to the primary cylinder 760, a bottom surface 1200 of the finger 980 slides along the spiraling ramped surface 960. The spring 680 pushes upwardly on outer cylinder 720 to keep a portion of the spiraling ramped upper surface 960 in contact with bottom surface 1200 of the finger 980 at all times. Enough friction exists between the focusing ring 480 and the main body 500 of the coupler 120 to prevent the spring 680 from rotating the focusing ring 480 when it is not being manipulated by a user. This friction makes the fine tuning of the focal length between the lens 200 and image sensor 140 (using focusing ring 480) relatively easy to accomplish.

FIGS. 9a and 9b illustrate the focusing mechanism at its two extreme focusing positions, with FIG. 9a illustrating the lens 200 at its closest position to the image sensor 140 and FIG. 9b illustrating the lens 200 at its furthest position from the image sensor 140. As shown in FIG. 9a, when the lens 200 is at its closest position to the image sensor 140, the spring 680 is fully compressed, bottom surface 1200 of finger 980 is in contact with a point 1060 near the top of the spiraling ramped surface 960, and the finger 980 is in a first position with respect to the primary cylinder 760. In contrast, as shown in FIG. 9b, when the lens 200 is at its furthest position from the image sensor 140, the spring 680 is fully extended, the bottom surface 1200 of finger 980 is in contact with a point 1040 near the bottom of the spiraling ramped surface 960, and the finger 980 is in a second position with respect to the primary cylinder 760, which is on an opposite side from the first position (FIG. 9a).

It should be appreciated that the above-described system for adjusting the focal length between the image sensor 140 and the lens 200 is only one example of the many possible systems that can achieve this result, as other implementations can alternatively be employed.

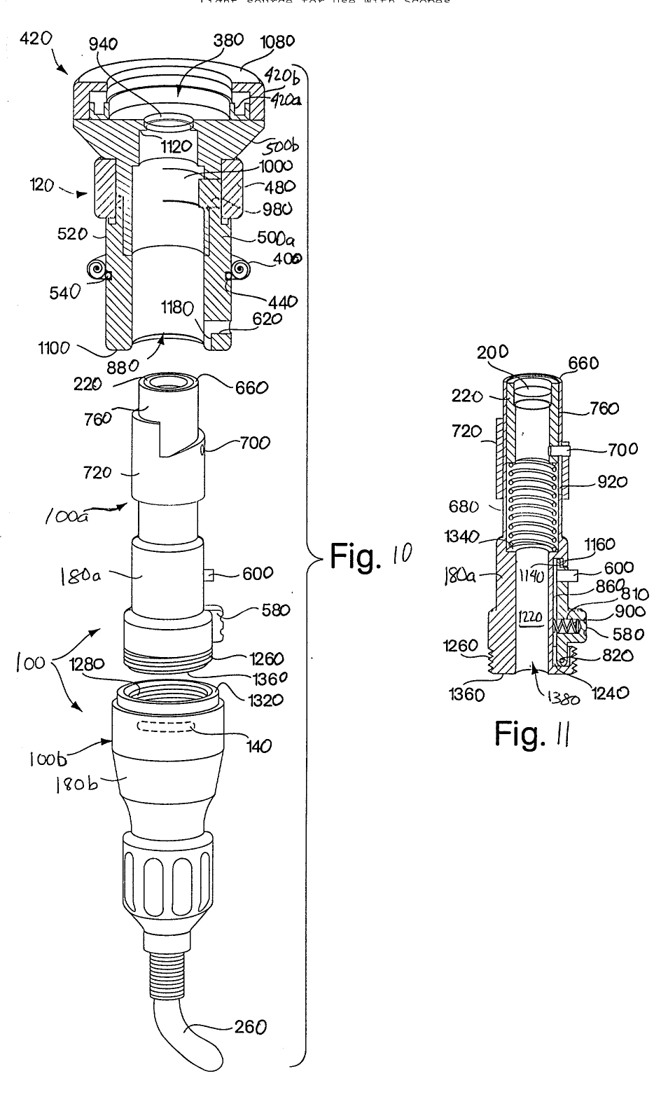

In the illustrative embodiment of FIGS. 7-8, the imaging unit 100 includes a single body portion 180 in which both the image sensor 140 (and associated circuitry 560) and the refractive lens 200 (and associated components such as the lens holder 220, the spring 680, and the cylinders 720 and 760) are disposed. It should be appreciated, however, that various components of the imaging unit 100 may alternatively be distributed among two or more separate housings that may be mated together to form the imaging unit 100. An illustrative example of an imaging system configured in this manner is shown in FIGS. 10 and 11. As shown in FIG. 10, the imaging unit 100 to be mated with the coupler 120 may include a first housing 180a in which the refractive lens (and associated components) is disposed, and a second housing 180b in which the image sensor 140 (and associated circuitry (not shown)) is disposed.

In the illustrative embodiment shown in FIGS. 10 and 11, the second housing 180b is the housing of a camera head 100b (e.g., a standard C-mount camera head), and the first housing 180a is the housing of an adapter 100a for adapting the camera head 100b for use with the coupler 120. When the adapter 100a is mated with the camera head 100b (as discussed below), the adapter 100a and the camera head 100b together form a composite imaging unit 100 which is similar to the imaging unit 100 described above in connection with FIGS. 7-8. Although the example shown in FIGS. 10-11 includes a C-mount camera head and adapter therefor, it should be appreciated that each of the housings 180a-180b may take on any of a number of alternative forms. For example, the housing 180b may alternatively be the housing of a standard V-mount camera head, or any other device in which an image sensor is disposed, and the housing 180a, may be configured to be mated with the same.

It should also be appreciated that the imaging unit 100 may further include additional housings, including only one or two housings. For example, referring to the FIG. 10 system, the imaging unit 100 may further include one or more housings disposed between the housings 180a and 180b or between the housing 180a and the coupler 120. Such an additional housing may exist, for example, in the form of a coupling device that couples together the housings 180a and 180b or the housing 180a and the coupler 120. It should be appreciated that the imaging unit actually employed may be any of numerous devices or combinations of devices capable of receiving an optical image along an imaging axis. As used herein, the term “imaging unit” is not intended to be limiting. Rather, it is intended to refer to any device or combination of devices capable of performing an imaging function.

Further, while in the systems of FIGS. 7-10 the coupler 120 is shown as being mated directly with the distal end 660 of the imaging unit 100, it should be appreciated that the imaging unit 100 may alternatively be mated indirectly with the coupler 120. For example, the imaging unit 100, in whatever form, may be mated with the coupler 120 via one or more additional coupling devices.

In the illustrative system shown in FIGS. 10-11, the operational interface between the adapter 100a and the coupler 120 is identical in most respects to the operational interface between the imaging unit 100 and the coupler 120 described above in connection with FIGS. 7-9. Corresponding components in the two embodiments have therefore been labeled with identical reference numerals, and reference may be made to the description of the embodiment of FIGS. 7-9 for an in-depth understanding of the operational interface between the adapter 100a and the coupler 120 of the embodiment of FIGS. 10-11.

As mentioned above, the camera head 100b may, for example, be a standard C-mount camera head. Therefore, as shown in FIG. 10, the camera head 100b may include a threaded, female connector 1280 formed at a distal end 1320 thereof. To permit the adapter 100a to mate with the connector 1280 of the camera head 100b, the adapter 100a may include a threaded, male connector 1260 formed at a proximal end 1360 thereof.

As shown in FIG. 10, the image sensor 140 may be disposed adjacent the distal end 1320 of the camera head 100b so that, when the male connector 1260 of the adapter 100a is threaded into the female connector 1280 of the camera head 100b, the image sensor 140 is disposed adjacent an opening 1380 at the proximal end 1360 of the adapter 100a. In the system of FIGS. 10-11, the image sensor 140 is therefore disposed further from the distal end 660 of the imaging unit 100 than it is in the system of FIGS. 7-8. For this reason, in the system of FIGS. 10-11, an annular cavity 1220 is formed within the housing 180a to provide an optical pathway between the refractive lens 200 and the image sensor 140 along which an image produced by the scope 10 (FIG. 7) can be focused onto the image sensor 140 via the lens 200. The cavity 1220 may be formed, for example, by reducing a width of an annular shoulder 1340 (FIG. 11) supporting one end of the spring 680 to be narrower than in the embodiment of FIGS. 7-8.

In addition, in the system of FIGS. 10-11, the button 580 is disposed on the adapter 100a of the imaging unit 100, and is therefore disposed distally of the image sensor 140 in this system, rather than proximally of the image sensor 140 as in the system of FIGS. 7-8. As shown, to make the button 580 fit on the adapter 100a, the button 580 may be shortened as compared to the system of FIGS. 7-9. Additionally, the pin 820 about which the button 580 pivots may be disposed within a small cavity 1240 adjacent the proximal end 1360 of the adapter 100a, rather than being disposed proximally of the image sensor 140 as in the system of FIGS. 7-9. It should be appreciated, of course, that the button 580 and locking member 600 represent only one example of numerous mechanisms that can be used to interconnect the imaging unit 100 with the coupler 120, and that the imaging unit 100 may be mated with the coupler 120 in different ways. For example, the imaging unit 100 may not include a button such as the button 580 or a locking member such as the locking member 600 at all, and may instead provide a different mechanism for mating the imaging unit 100 with the coupler 120.

In light of the above description, it should be appreciated that, as far as the physical interface between the imaging unit 100 and the coupler 120 is concerned, the imaging unit 100 that is formed when the adapter 100a is mated with the camera head 100b can be made identical in all respects to the imaging unit 100 of embodiment of FIGS. 7-9. Additionally, by properly adjusting the refractive index of the lens 200 to account for the increased distance between the distal end 660 and the image sensor 140 in the embodiment of FIGS. 10-11 as compared to the embodiment of FIGS. 7-9, the imaging unit 100 of FIGS. 10-11 can also be made to mimic the functional characteristics of the imaging unit 100 of FIGS. 7-9 as well. The use of the adapter 100a of FIGS. 10-11 therefore enables a standard camera head (e.g., the camera head 100b) to be adapted for use with the inventive coupler 120 described herein in the same manner as in the embodiment of the imaging unit 100 described in connection with FIGS. 7-9. Therefore, one already in possession of a camera head 100b (e.g., a standard C-mount or V-mount camera head) may simply purchase the adapter 100a (which does not include an image sensor) for use with the coupler 120, rather than purchasing the imaging unit 100 of FIGS. 7-9 (which additionally includes an image sensor) for use therewith.

The adapter 100a described herein is configured for use with a specific type of coupler (i.e., the coupler 120). However, it should be appreciated that the adapter 100a may alternatively be configured for use with other types of devices or couplers.

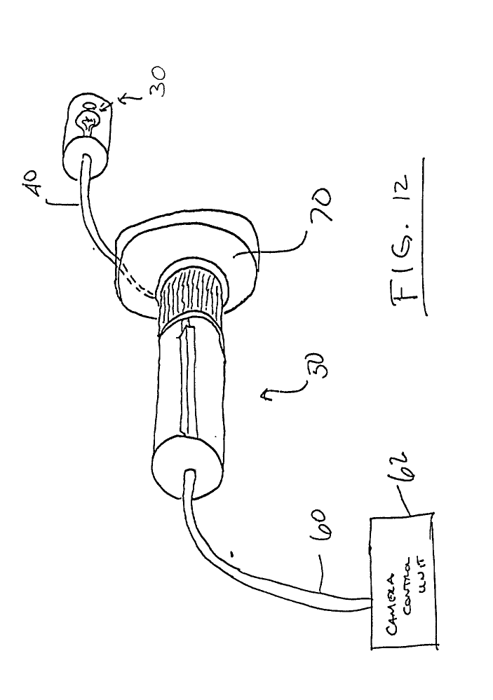

In another variation, as shown in FIG. 12, the light source assembly 30 may be an integral part of a coupling device 70, such as coupler 120 discussed with reference to FIGS. 7-11, used to couple the camera assembly to the scope. The light source assembly may be attached to the coupling device 70 via cord 40. The power source (e.g., battery) to the light source may be disposed within the coupling device. Alternatively, the coupling device may include a suitable connector so that a power connection may be made between the light source assembly 30 and the camera assembly 50.

The above-mentioned features of the present invention, whether in the combinations described, or in other suitable combinations, provide a number of advantages. For example, the present invention includes a light source assembly that is less than five pounds. Preferably, the light source assembly is less than one pound. More preferably, the light source assembly is less than one-half pound and most preferably, less than one-quarter pound. With such a lightweight light source assembly, increased mobility and maneuverability, among other advantages, may be attained. Increased mobility and maneuverability, as well as other advantages, may also be attained with the use of a self-contained and other light source assemblies as described above.

It should be appreciated that various combinations of the above-described embodiments of the present invention can be employed together, but each aspect of the present invention can be used separately. Therefore, although the specific embodiments disclosed in the figures and described in detail employ particular combinations of the above-discussed features of the present invention, it should be appreciated that the present invention is not limited in this respect, as the various aspects of the present invention can be employed separately, or in different combinations. Thus, the particular embodiments described in detail are provided for illustrative purposes only.

What is claimed is:

1. An apparatus comprising:
a scope comprising:
a body having a proximal end and a distal end;
a light input port disposed on the body;
a light output port disposed on the body and disposed distally of the light input port; and
a light transmission channel extending between the light input port and the light output port; and
a light source assembly comprising:
a housing including a light output port, the housing being mounted to the body with the light output port of the housing in optical communication with the light input port on the body; and
at least one light source disposed within the housing.

2. The scope according to claim 1, wherein the housing is integrally formed with the body of the scope.

3. The scope according to claim 1, wherein the at least one light source is selected from the group consisting of any one or combination of light-emitting diodes, halogen bulbs, and xenon bulbs.

4. The scope according to claim 1, wherein the at least one light source comprises at least one light-emitting diode.

5. The scope according to claim 1, wherein the at least one light-emitting source comprises at least one incandescent light bulb.

6. The scope according to claim 1, wherein the housing is vented.

7. The scope according to claim 1, wherein the light source assembly further comprises a fan to cool the light source assembly.

8. The scope according to claim 1, wherein the housing is constructed and arranged to transmit light directly to the light input port of the scope without allowing light to otherwise escape from the housing.

9. The scope according to claim 1, wherein the housing includes at least one portion that is movable to provide access to the light source to facilitate changing the light source.

10. The scope according to claim 1, wherein the light source assembly is cordless.

11. The scope according to claim 1, wherein the light source assembly includes a battery that powers the at least one light source.

12. The scope according to claim 11, wherein the battery is disposed within the housing.

13. The scope according to claim 11, wherein the battery is disposed in a power pack that is separate from the housing.

14. The scope according to claim 1, wherein the light source assembly further comprises a user interface coupled to the light source to selectively activate the light source.

15. A light source assembly for use with a scope, the scope including a body having a proximal end and a distal end, a light input port disposed on the body, a light output port disposed on the body and disposed distally of the light input port, and a light transmission channel extending between the light input port and the light output port, the light source assembly comprising:
a housing including an interface to mount the housing to the body of the scope, the housing further including a light output port that is adapted to mate with the light input port on the body when the interface engages with the body; and
at least one light source disposed within the housing.

16. The light source assembly according to claim 15, wherein the at least one light source is selected from the group consisting of any one or combination of light-emitting diodes, halogen bulbs, and xenon bulbs.

17. The light source assembly according to claim 15, wherein the at least one light source comprises at least one light-emitting diode.

18. The light source assembly according to claim 15, wherein the at least one light source comprises at least one incandescent light bulb.

19. The light source assembly according to claim 15, wherein the housing is vented.

20. The light source assembly according to claim 15, further comprising a fan to cool the light source assembly.

21. The light source assembly according to claim 15, wherein the housing is constructed and arranged to transmit light directly to the light input port of the scope without allowing light to otherwise escape from the housing.

22. The light source assembly according to claim 15, wherein the housing includes at least one portion that is movable to provide access to the light source to facilitate changing the light source.

23. The light source assembly according to claim 15, wherein the light source assembly is cordless.

24. The light source assembly according to claim 15, wherein the light source assembly includes a battery that powers the at least one light source.

25. The light source assembly according to claim 24, wherein the battery is disposed within the housing.

26. The light source assembly according to claim 24, wherein the battery is disposed in a power pack that is separate from the housing.

27. The light source assembly according to claim 15, further comprising a user interface coupled to the light source to selectively activate the light source.

28. The light source assembly according to claim 15, wherein the light source assembly is less than five pounds.

29. The light source assembly according to claim 28, wherein the light source assembly is less than one pound.

30. The light source assembly according to claim 29, wherein the light source assembly is less than one-half pound.

31. The light source assembly according to claim 30, wherein the light source assembly is less than one-quarter pound.

32. A coupler for coupling a light source assembly to a scope, the scope including a body having a proximal end and a distal end, a light input port disposed on the body, a light output port disposed on the body and disposed distally of the light input port, and a light transmission channel extending between the light input port and the light output port, the light source assembly including a housing having a light output port, and at least one light source disposed within the housing, the coupler comprising:
a coupler body;
first and second interfaces formed on the coupler body; and
a light transmission channel in optical communication with the first and second interfaces;
wherein the first interface is adapted to be mounted to the light source assembly and align with the light output port on the light source assembly and wherein the second interface is adapted to be mounted to the scope and align with the light input port on the scope such that light emission from the light source is transmitted to the scope through the interface.

33. The coupler according to claim 32, in combination with the light source assembly.

34. The coupler according to claim 32, in combination with the scope.

35. The combination according to claim 33, in combination with the scope.

36. The combination according to claim 33, wherein the at least one light source is selected from the group consisting of any one or combination of light-emitting diodes, halogen bulbs, and xenon bulbs.

37. The combination according to claim 33, wherein the at least one light source comprises at least one light-emitting diode.

38. The combination according to claim 33, wherein the at least one light source comprises at least one incandescent light bulb.

39. The combination according to claim 33, wherein the housing is vented.

40. The combination according to claim 33, wherein the light source assembly further comprises a fan to cool the light source assembly.

41. The coupler according to claim 32, wherein the coupler body is vented.

42. The combination according to claim 33, wherein the light source assembly further comprises a fan adapted to cool the light source assembly.

43. The combination according to claim 33, wherein at least one of the light source assembly and the coupler is constructed and arranged to transmit light directly to light input port of the scope without allowing light to otherwise escape from the at least one of the housing and the coupler body.

44. The coupler according to claim 32, wherein the coupler body is constructed and arranged to transmit light from the light source assembly directly to the light input port of the scope without allowing light to otherwise escape from the coupler body.

45. The combination according to claim 33, wherein the housing of the light source assembly includes at least one portion that is movable to provide access to the light source to facilitate changing the light source.

46. The combination according to claim 33, wherein the light source assembly is cordless.

47. The combination according to claim 33, wherein the light source assembly includes a battery that powers the at least one light source.

48. The combination according to claim 47, wherein the battery is disposed within the housing of the light source assembly.

49. The combination according to claim 47, wherein the battery is disposed in a power pack that is separate from the housing of the light source assembly.

50. The combination according to claim 33, wherein the light source assembly further comprises a user interface coupled to the light source to selectively activate the light source.

51. The coupler according to claim 32, further comprising a power source adapted to power the light source assembly when the coupler is coupled to the light source assembly.

52. The coupler according to claim 51, further comprising a user interface adapted to selectively activate the light source.

53. A camera system for use with a light source assembly and a scope, the scope including a body having a proximal end and a distal end, a light input port disposed on the body, a light output port disposed on the body and disposed distally of the light input port and a light transmission channel extending between the light input port and the light output port, the light source assembly including a housing, having a light output port, and at least one light source disposed within the housing, the camera system comprising:
a camera controller, coupled to the camera, to control the camera, the camera controller comprising an interface adapted to further couple to the light source assembly, the controller adapted to control the light source assembly.

54. The camera system according to claim 53, in combination with the light source assembly.

55. The camera system according to claim 53, in combination with the scope.

56. The combination according to claim 54, in combination with the scope.

57. The combination according to claim 54, wherein the at least one light source is selected from the group consisting of any one or combination of light-emitting diodes, halogen bulbs, and xenon bulbs.

58. The combination according to claim 54, wherein the at least one light source comprises at least one light-emitting diode.

59. The combination according to claim 54, wherein the at least one light comprises at least one incandescent light bulb.

60. The combination according to claim 54, wherein the housing of the light source assembly is vented.

61. The combination according to claim 54, wherein the light source assembly further comprises a fan to cool the light source assembly.

62. The combination according to claim 54, wherein the housing of the light source assembly is constructed and arranged to transmit light directly to the light input port of the scope without allowing light to otherwise escape from the housing.

63. The combination according to claim 54, wherein the housing of the light source assembly includes at least one portion that is movable to provide access to the light source to facilitate changing the light source.

64. The combination according to claim 54, wherein the light source assembly includes a battery that powers the at least one light source.

65. The combination according to claim 64, wherein the battery is disposed within the housing of the light source assembly.

66. The camera system according to claim 53, further comprising a power source adapted to power the light source assembly.

67. The camera system according to claim 53, wherein the camera system is cordless.

68. The camera system according to claim 53, wherein the camera comprises a charge couple device.

69. The camera system according to claim 53, wherein the camera controller is disposed outside the camera body.

70. The camera system according to claim 53, wherein the camera controller is adapted to at least one of display, store and transmit data or images.

71. A camera coupler for use with a scope, the scope including a body having a proximal end and a distal end, a light input port disposed on the body, a light output port disposed on the body and disposed distally of the light input port, a light transmission channel extending between the light input port and the light output port, and a view port, the camera coupler comprising:
a coupler body adapted to couple the camera to the view port; and
a light source assembly connected to the coupler body and being adapted to couple with the light input port of the scope.

72. The camera coupler according to claim 71, wherein the light source assembly includes a housing having a light output port and at least one light source disposed within the housing.

73. The camera coupler according to claim 71, in combination with the scope.

74. The combination according to claim 72, in combination with the scope.

75. The combination according to claim 72, wherein the at least one light source is selected from the group consisting of any one or combination of light-emitting diodes, halogen bulbs, and xenon bulbs.

76. The combination according to claim 72, wherein the at least one light source comprises at least one light-emitting diode.

77. The combination according to claim 72, wherein the at least one light source comprises at least one incandescent light bulb.

78. The combination according to claim 72, wherein the housing of the light source assembly is vented.

79. The combination according to claim 72, wherein the light source assembly further comprises a fan to cool the light source assembly.

80. The combination according to claim 72, wherein the housing is constructed and arranged to transmit light directly to the light input port of the scope without allowing light to otherwise escape from the housing.

81. The combination according to claim 72, wherein the housing includes at least one portion that is movable to provide access to the light source to facilitate changing the light source.

82. The combination according to claim 72, wherein the light source assembly includes a battery that powers the at least one light source.

83. The combination according to claim 82, wherein the battery is disposed within the housing.

84. The camera coupler according to claim 71, further comprising a power source adapted to power the light source assembly.

85. The camera coupler according to claim 84, wherein the power source is a battery.

86. The camera coupler according to claim 84, further comprising a cord connection between the coupler body and the light source assembly.

87. The combination according to claim 72, further comprising a battery disposed within the coupler body and a cord connection disposed between the coupler and the light source assembly for delivering power from the battery to the light source.

88. A method for providing light to a scope, the method comprising the acts of:
providing a scope having a body, the body having a proximal end and a distal end, a light input port disposed on the body, a light output port disposed on the body and disposed distally of the light input port and a light transmission channel extending between the light input port and the light output port;
providing a light source assembly having a housing, including a light output port, and a light source disposed within the housing; and
mating the light source assembly to the scope, with the light output port of the light source assembly aligning with the light input port of the scope.

89. The method according to claim 88, further comprising the act of powering the light source with a battery.

90. The method according to claim 88, further comprising the act of transmitting light from the light source directly to the scope without allowing any light to otherwise escape from the housing of the light source assembly.

91. The method according to claim 88, further comprising the act of cooling the light source assembly.

92. The method according to claim 88, wherein the act of mating the light source assembly to the scope comprises the act of integrally forming the light source assembly with the scope.

93. The method according to claim 88, wherein the act of mating the light source assembly to the scope comprises the act of mounting a coupler between the light source assembly and the scope.

94. The method according to claim 88, further comprising the act of coupling a camera system, having a camera and a camera controller, to the light source assembly.

95. The method according to claim 94, further comprising the act of controlling the light source with the camera controller.

96. The method according to claim 95, wherein the act of controlling the light source further comprises the act of selectively activating the light source.
